(19) United States
(12) Patent Application Publication
(10) Pub. No.: US 2024/0001356 A1
(43) Pub. Date: Jan. 4, 2024

(54) METHOD FOR TREATING A CATALYST BEFORE UNLOADING

(71) Applicant: ARKEMA FRANCE, Colombes (FR)

(72) Inventors: Laurent WENDLINGER, PIERRE-BENITE CEDEX (FR); Dominique DEUR-BERT, PIERRE-BENITE CEDEX (FR)

(21) Appl. No.: 18/036,438

(22) PCT Filed: Nov. 24, 2021
(86) PCT No.: PCT/FR2021/052079

§ 371 Date: May 11, 2023

(30) Foreign Application Priority Data
Nov. 27, 2020 (FR) 2012271

(51) Int. Cl. B01J 38/04; B01J 23/86; C07C 17/20

(52) U.S. Cl. CPC B01J 38/04; B01J 23/866; C07C 17/206

(57) ABSTRACT

The present invention relates to a process for treating, in a reactor containing a catalytic bed, a solid catalyst, said process comprising the steps of: a) implementing, in said reactor, a gas-phase catalytic reaction at a catalytic bed temperature T1 in the presence of a hydrogen halide or giving rise to the formation of a hydrogen halide, b) causing an inert gas to flow through the catalytic bed at a catalytic bed temperature T2 that is lower than T1, the temperature T2 being greater than 30° C.

### TECHNICAL FIELD OF THE INVENTION

The present invention relates to a process for treating a catalyst. In particular, the present invention relates to a process for treating a catalyst before it is unloaded.

### TECHNOLOGICAL BACKGROUND OF THE INVENTION

The catalysts used in chemical reactions are essential materials for the production of many compounds, and have the main function of increasing the rate of the reactions. The catalysts are selected according to the reaction that is to be carried out.

In the field of the production of alkane or alkene compounds containing halogen atoms, the catalyst is generally used in gas phase. The catalyst may either be used in combination with a hydrogen halide, or give rise to a hydrogen halide during the implementation of a reaction. At the end of the implementation of the reaction, some of the hydrogen halide is present in the reactor and even absorbed or adsorbed by the catalyst itself. The presence of this hydrogen halide is particularly dangerous when there is a need to perform maintenance operations that necessitate the unloading of the catalyst.

It is therefore necessary to remove this hydrogen halide from the reactor and from the catalyst in order to guarantee the safety of intervening personnel during the unloading of the catalyst. The document EP 3 238 820 describes a process for unloading a catalyst implementing a step of high-temperature treatment. This high-temperature treatment step has a tendency to partially degrade the catalyst, and consumes a great deal of energy.

There is therefore a need for a catalyst unloading process that is safe, effective and inexpensive in terms of energy.

### SUMMARY OF THE INVENTION

The present invention relates to a process for treating, in a reactor containing a catalytic bed, a solid catalyst, said process comprising the steps of:
a) implementing, in said reactor, a gas-phase catalytic reaction at a catalytic bed temperature T1 in the presence of a hydrogen halide or giving rise to the formation of a hydrogen halide,
b) causing an inert gas to flow through the catalytic bed at a catalytic bed temperature T2 that is lower than T1, the temperature T2 being greater than 30° C.

The present process provides an economic and energetic saving since the inert gas flows through the catalytic bed at a temperature that is lower than the temperature of the catalytic reaction. In addition, by implementing step b) at a temperature T2 that is lower than the temperature T1 at which the catalytic reaction of step a) is implemented, the catalyst does not deteriorate, which enables subsequent use of the latter, optionally after regeneration, without a loss in activity. In the present process, the implementation of step b) is after, preferably subsequent to, the implementation of step a).

According to a preferred embodiment, the inert gas introduced into the reactor is at a temperature of between ambient temperature and the temperature T2. Preferably, the inert gas introduced into the reactor is at ambient temperature. The inert gas introduced will thus gradually cool the catalytic bed while removing the hydrogen halide residues present in the reactor and adsorbed or absorbed in the catalyst. The introduction of an inert gas having a temperature of ambient temperature or close to ambient temperature enables an additional energetic and economic saving.

According to a preferred embodiment, the hydrogen halide is hydrogen fluoride or hydrogen chloride.

According to a preferred embodiment, the temperature T2 decreases during the implementation of step b), preferably the temperature T2 decreases at a rate of less than 1° C./min during the implementation of step b).

According to a preferred embodiment, the inert gas flows at a flow rate of greater than 0.1 ml/min per ml of catalyst.

According to a preferred embodiment, the catalyst is based on carbon or based on a metal selected from the group consisting of Cr, Fe, Sb, Ni, Co, Zn, Al and Mn.

According to a preferred embodiment, step a) implements a gas-phase reaction between HF and a C₁-C₄ halohydrocarbon compound A or step a) implements a gas-phase dehydrohalogenation reaction of a saturated C₁-C₄ hydrocarbon compound B comprising at least one halogen atom to form an unsaturated C₁-C₄ hydrocarbon compound and a hydrogen halide.

According to a preferred embodiment, the compound A is selected from the group consisting of 1,1,2-trichloroethane, 2-chloro-1,1,1-trifluoroethane, 1-chloro-1,1,2-trifluoroethane, 1-chloro-1,2,2-trifluoroethane, 1,1,1,3,3,3-hexachlorodifluoropropane, 1,1,1,3,3,3-hexachloropropane, 1,1,1,3,3-pentachloropropane, 2,2,3-trichloro-1,1,1,3,3-pentafluoropropane, 1,1,1,3,3,3-hexachlorodifluoropropane, 1,1-dichloro-2,2,3,3,3-pentafluoropropane, 1,3-dichloro-1,2,2,3,3-pentafluoropropane, 1,1,1,2,3-pentachloropropane, 1,1,2,2,3-pentachloropropane, 1,1,1,3,3-pentachloropropane, 1,2-dichloroethylene, 1,1,2-trichloroethylene, 1,1,2,2-tetrachloroethylene, 1,1,2-trichloro-3,3,3-trifluoropropene, hexafluoropropene, 1,1,3,3,3-pentafluoropropene, 1,3,3,3-tetrafluoropropene, 2-chloro-3,3,3-trifluoropropene, 1,1,2,3-tetrachloropropene, 2,3,3,3-tetrachloropropene, 1-chloro-3,3,3-trifluoropropene, 1,1,3,3-tetrachloropropene, 1,3,3,3-tetrachloropropene, 2,3-dichloro-1,1,1-trifluoropropane, 2-chloro-1,1,1,2-tetrafluoropropane; or
the compound B is selected from the group consisting of 1,1-difluoroethane, 1,1,1-trifluoroethane, 1,1,1,2,2-pentafluoroethane, 2-chloro-1,1,1-trifluoroethane, 1,1,1,2-tetrafluoroethane, 1,1,2,2-tetrafluoroethane, 1,1,1,2-tetrafluoropropane, 1,1,1,2,3,3-hexafluoropropane, 1,1,1,3,3,3-hexafluoropropane, 1,1,1,2,2,3-hexafluoropropane, 1,1,1,2,2-pentafluoropropane, 1,1,1,2,3-pentafluoropropane, 1,1,1,3,3-pentafluoropropane, 2,3-dichloro-1,1,1-trifluoropropane, 2-chloro-1,1,1,2-tetrafluoropropane.

According to a preferred embodiment, said process comprises a step c) of unloading the catalyst from said reactor.

According to a preferred embodiment, step b) comprises a step b1) of cooling the catalytic bed from the temperature T1 to T2, and then a step b2) of causing said inert gas to flow through the catalytic bed.

The present invention also relates to a process for treating, in a reactor containing a catalytic bed, a solid catalyst, said process comprising the steps of:
a) implementing, in said reactor, a gas-phase catalytic reaction at a catalytic bed temperature T1 in the presence of a hydrogen halide or giving rise to the formation of a hydrogen halide,
b) causing an inert gas to flow through the catalytic bed at a catalytic bed temperature T2 that is lower than T1, the temperature T2 decreasing during the implementation of step b).

### DETAILED DESCRIPTION OF THE PRESENT INVENTION

The present invention relates to a process for treating a catalyst. In particular, the present invention relates to a process for treating a solid catalyst. Thus, the present invention relates to a process for treating, in a reactor containing a catalytic bed, a solid catalyst.

Preferably, the present process comprises the steps of:
a) implementing, in said reactor, a gas-phase catalytic reaction at a catalytic bed temperature T1 in the presence of a hydrogen halide or giving rise to the formation of a hydrogen halide,
b) causing an inert gas to flow through the catalytic bed at a catalytic bed temperature T2 that is lower than T1, the temperature T2 being greater than 30° C.

The process according to the present invention is typically conducted in a reactor provided with a fixed catalytic bed. The reactor and its associated feed lines, effluent lines and associated devices have to be constructed from materials which are resistant to hydrogen halides such as hydrogen fluoride or hydrogen chloride. Typical construction materials, well known in the state of the art of fluorination, include stainless steels, in particular of austenitic type, well-known alloys having a high nickel content, such as Monel© nickel/copper alloys, Hastelloy© nickel-based alloys and Inconel© nickel/chromium alloys.

According to a preferred embodiment, in step a) the hydrogen halide is in anhydrous form. Preferably, the compound A and the compound B described above can also be in anhydrous form.

According to a preferred embodiment, in step b) the inert gas is in anhydrous form.

The term anhydrous refers to a content by mass of water of less than 1000 ppm, advantageously 500 ppm, preferably of less than 200 ppm, in particular of less than 100 ppm, more particularly of less than 50 ppm and favorably of less than 25 ppm, in the compound under consideration.

Catalyst

According to a preferred embodiment, the catalyst is based on carbon or on a metal selected from the group consisting of Cr, Ti, Al, Mn, Ni, Co, Fe, Cu, Zn, Sn, Au, Ag, Pt, Pd, Ru, Rh, Mo, Zr, Ge, Nb, Ta, Ir, Hf, V, Mg, Li, Na, K, Ca, Cs, Ru and Sb; preferably, the catalyst is based on a metal selected from the group consisting of Cr, Fe, Sb, Ni, Co, Zn, Al et Mn. The carbon-based catalyst may be activated carbon, charcoal or graphite. The metal-based catalyst may be in the oxide, halide, or oxyhalide form of said metal. In particular, the catalyst is based on Cr, Al, Fe or Sb. The catalyst may be an antimony, iron or aluminum halide, such as SbCl₅, FeCl₃, or AlCl₃. The catalyst may be a chromium oxide, a chromium oxyfluoride or a chromium fluoride. When the catalyst is based on chromium, it may contain a cocatalyst selected from the group consisting of Co, Zn, Mn, Ni or a mixture thereof, in a content by mass of from 1% to 10% based on the total weight of said catalyst.

Said catalyst may be a bulk or supported catalyst. The support may be selected from the group consisting of activated carbon, alumina and aluminum fluoride. For example, catalysts such as Cr₂O₃, MgF₂, SbCl₅ or FeCl₃ may be supported on activated carbon.

Said catalyst may be activated before implementing the step a) detailed below. The catalyst may be activated according to the methods known to those skilled in the art. For example, the catalyst may be activated in the presence of oxygen, of HF or of nitrogen, or a mixture thereof, at a temperature of between 100° C. and 500° C.

Step a)

Said step a) comprises implementing, in said reactor, a gas-phase catalytic reaction at a catalytic bed temperature T1. Said catalytic reaction may either be implemented in the presence of a hydrogen halide or give rise to the formation of a hydrogen halide.

The hydrogen halide may be selected from the group consisting of HF, HCl, HBr and HI. Preferably, the hydrogen halide is hydrogen fluoride (HF) or hydrogen chloride (HCl).

According to a preferred embodiment, step a) may implement a gas-phase reaction between HF and a C₁-C₄ halohydrocarbon compound A. Preferably, step a) implements a reaction between hydrogen fluoride and a compound A to form a halohydrocarbon compound comprising at least one fluorine atom. Said compound A may be a saturated compound of the formula CH₂Cl₂, CH₂Br₂, CHCl₃, CCl₄, C₂Cl₆, C₂BrCl₅, C₂Cl₅F, C₂Cl₄F₂, C₂Cl₃F₃, C₂Cl₂F₄, C₂ClF₅, C₂HCl₅, C₂HCl₄F, C₂HCl₃F₂, C₂HCl₂F₃, C₂HClF₄, C₂HBrF₄, C₂H₂Cl₄, C₂H₂Cl₃F, C₂H₂Cl₂F₂, C₂H₂ClF₃, C₂H₃Cl₃, C₂H₃Cl₂F, C₂H₃ClF₂, C₂H₄Cl₂, C₂H₄ClF, C₃Cl₆F₂, C₃Cl₅F₃, C₃Cl₄F₄, C₃Cl₃F₅, C₃HCl₇, C₃HCl₆F, C₃HCl₅F₂, C₃HCl₄F₃, C₃HCl₃F₄, C₃HCl₂F₅, C₃Cl₂F₆, C₃H₂Cl₆, C₃H₂BrCl₅, C₃H₂Cl₅F, C₃Cl₄F₂, C₃H₂Cl₃F₃, C₃H₂Cl₂F₄, C₃H₂ClF₅, C₃H₃Cl₅, C₃H₃Cl₄F, C₃H₃Cl₃F₂, C₃H₃Cl₂F₃, C₃H₃ClF₄, C₃H₄Cl₄, C₄H₄Cl₄, C₄H₄Cl₆, C₄H₆Cl₆, C₄H₅Cl₄F₁ or C₆H₄Cl₈, or an unsaturated compound of the formula C₂Cl₄, C₂BrCl₃, C₂Cl₃F, C₂Cl₂F₂, C₂ClF₃, C₂F₄, C₂HCl₃, C₂HBrCl₂, C₂HCl₂F, C₂HClF₂, C₂HF₃, C₂H₂Cl₂, C₂H₂ClF, C₂H₂F₂, C₂H₃Cl, C₂H₃F, C₂H₄, C₃H₆, C₃H₅Cl, C₃H₄C₂, C₃H₃Cl₃, C₃H₂Cl₄, C₃HCl₅, C₃H₂ClF₃, C₃F₃HCl₂, C₃F₂H₂Cl₂, C₃F₄H, ClC₃Cl₆, C₃Cl₅F, C₃Cl₄F₂, C₃Cl₃F₃, C₃Cl₂F₄, C₃ClF₅, C₃HF₅, C₃H₂F₄, C₃F₆, C₄Cl₈, C₄Cl₂F₆, C₄ClF₇, C₄H₂F₆, or C₄HClF₆.

Preferably, said compound A may be selected from the group consisting of 1,1,2-trichloroethane, 2-chloro-1,1,1-trifluoroethane, 1-chloro-1,1,2-trifluoroethane, 1-chloro-1,2,2-trifluoroethane, 1,1,1,3,3,3-hexachlorodifluoropropane, 1,1,1,3,3,3-hexachloropropane, 1,1,1,3,3-pentachloropropane, 2,2,3-trichloro-1,1,1,3,3-pentafluoropropane, 1,1,1,3,3,3-hexachlorodifluoropropane, 1,1-dichloro-2,2,3,3,3-pentafluoropropane, 1,3-dichloro-1,2,2,3,3-pentafluoropropane, 1,1,1,2,3-pentachloropropane, 1,1,2,2,3-pentachloropropane, 1,1,1,3,3-pentachloropropane, 1,2-dichloroethylene, 1,1,2-trichloroethylene, 1,1,2,2-tetrachloroethylene, 1,1,2-trichloro-3,3,3-trifluoropropene, hexafluoropropene, 1,1,3,3,3-pentafluoropropene, 1,3,3,3-tetrafluoropropene, 2-chloro-3,3,3-trifluoropropene, 1,1,2,3-tetrachloropropene, 2,3,3,3-tetrachloropropene, 1-chloro-3,3,3-trifluoropropene, 1,1,3,3-tetrachloropropene, 1,3,3,3-tetrachloropropene, 2,3-dichloro-1,1,1-trifluoropropane, 2-chloro-1,1,1,2-tetrafluoropropane.

More preferentially, said compound A may be selected from the group consisting of 1,1,2-trichloroethane, 2-chloro-1,1,1-trifluoroethane, 1-chloro-1,1,2-trifluoroethane, 1-chloro-1,2,2-trifluoroethane, 2-chloro-3,3,3-trifluoropropene, 1,1,1,2,3-pentachloropropane, 1,1,2,2,3-pentachloropropane, 1,1,2,3-tetrachloropropene, 2,3,3,3-tetrachloropropene, 1-chloro-3,3,3-trifluoropropene, 1,1,1,3,3-pentachloropropane, 1,1,3,3-tetrachloropropene, 1,3,3,3-tetrachloropropene, 2,3-dichloro-1,1,1-trifluoropropane, 2-chloro-1,1,1,2-tetrafluoropropane, or mixtures thereof.

In particular, specific examples of reaction between HF and the compound A include the conversion of 1,1,2-trichloroethane (CHCl₂CH₂Cl or HCC-140) into 1-chloro-2,2-difluoroethane (CH₂ClCF₂H or HCFC-142), the conversion of 1,1,1,3,3,3-hexachlorodifluoropropane (CCl₃CF₂CCl₃ or CFC-212ca) into a mixture of 1,1,3-trichloro-1,2,2,3,3-pentafluoropropane (CCl₂FCF₂CClF₂ or CFC-215ca) and 1,3-dichloro-1,1,2,2,3,3-hexafluoropropane (CClF₂CF₂CClF₂ or CFC-216ca), the conversion of 1,1,1,3,3,3-hexachloropropane (CCl₃CH₂CCl₃ or HCC-230fa) into 1-chloro-1,1,3,3,3-pentafluoropropane (CF₃CH₂CClF₂ or HCFC-235fa) and 1,1,1,3,3,3-hexafluoropropane (CF₃CH₂CF₃ or HFC-236fa), the conversion of 1,1,1,3,3-pentachloropropane (CCl₃CH₂CHCl₂ or HCC-240fa) into a mixture of 1,1,1,3,3-pentafluoropropane (CHF₂CH₂CF₃ or HFC-245fa), 1-chloro-3,3,3-trifluoro-1-propene (CHCl═CHCF₃ or HCFO-1233zd) and 1,3,3,3-tetrafluoropropene (CHF═CHCF₃ or HFO-1234ze), the conversion of 2,2,3-trichloro-1,1,1,3,3-pentafluoropropane (CF₃CCl₂CClF₂ or CFC-215aa) into a mixture of 1,1,1,3,3,3-hexachlorodifluoropropane (CF₃CCl₂CF₃ or CFC-212ca) and 2-chloro-1,1,1,2,3,3,3-heptafluoropropane (CF₃CClFCF₃ or CFC-217ba), the conversion of 1,1,1,3,3,3-hexachlorodifluoropropane (CF₃CCl₂CF₃ or CFC-212ca) into 2-chloro-1,1,1,2,3,3,3-heptafluoropropane (CF₃ClFCF₃ or CFC-217ba), the conversion of a mixture containing 1,1-dichloro-2,2,3,3,3-pentafluoropropane (CF₃CF₂CHCl₂ or HCFC-225ca) and 1,3-dichloro-1,2,2,3,3-pentafluoropropane (CClF₂CF₂CHClF or HCFC-225cb) into a mixture of 1-chloro-1,2,2,3,3,3-hexafluoropropane (CF₃CF₂CHClF or HCFC-226ca) and 1,1,1,2,2,3,3-heptafluoropropane (CF₃CF₂CHF₂ or HFC-227ca), the conversion of 1,1,1,2,3-pentachloropropane (CCl₃CHClCH₂Cl or HCC-240db) into 2-chloro-3,3,3-trifluoro-1-propene (CF₃CCl═CH₂ or HCFO-1233xf), the conversion of 1,1,2,2,3-pentachloropropane (CHCl₂CCl₂CH₂Cl or HCC-240aa) into 2-chloro-3,3,3-trifluoro-1-propene (CF₃CCl═CH₂ or HCFO-1233xf), the conversion of 1,1,1,2,3-pentachloropropane (CCl₃CHClCH₂Cl or HCC-240db) into 2,3,3,3-tetrafluoropropene (CF₃CF═CH₂ or HFO-1234yf), the conversion of 1,1,2,2,3-pentachloropropane (CHCl₂CCl₂CH₂Cl or HCC-240aa) into 2,3,3,3-tetrafluoropropene (CF₃CF═CH₂ or HFO-1234yf), the conversion of 1,1,1,3,3-pentachloropropane (CCl₃CH₂CHCl₂ or HCC-240fa) into 1,3,3,3-tetrafluoropropene (CF₃CH═CHF or HFO-1234ze), the conversion of 1,2-dichloroethylene (CHCl═CClH or HCO-1130) into 1-chloro-2,2-difluoroethane (CH₂ClCF₂H or HCFC-142)₂, the conversion of 1,1,2-trichloro-3,3,3-trifluoro-1-propene (CCl₂═CClCF₃ or CFC-1213xa) into a mixture of 2,3-dichloro-1,1,1,3,3-pentafluoropropane (CF₃CHClCClF₂ or HCFC-225da), of 2-chloro-1,1,1,3,3,3-hexafluoropropane (CF₃CHClCF₃ or HCFC-226da) and/or of 2-chloro-1,1,3,3,3-pentafluoro-1-propene (CF₃CCl═CF₂ or CFC-1215xc), the conversion of hexafluoropropene (CF₃CF═CF₂ or CFC-1216yc) into 1,1,1,2,3,3,3-heptafluoropropane (CF₃CHFCF₃ or HFC-227ea), the conversion of 1,1,3,3,3-pentafluoropropene (CF₃CH═CF₂ or HFO-1225zc) into 1,1,1,3,3,3-hexafluoropropane (CF₃CH₂CF₃ or HFC-236fa), the conversion of 1,3,3,3-tetrafluoropropene (CF₃CH═CHF or HFO-1234ze) into 1,1,1,3,3-pentafluoropropane (CF₃CH₂CHF₂ or HFC-245fa), the conversion of 2-chloro-3,3,3-trifluoro-1-propene (CF₃CCl═CH₂ or HCFO-1233xf) into 2,3,3,3-tetrafluoropropene (CF₃CF═CH₂ or HFO-1234yf), the conversion of 1,1,2,3-tetrachloro-1-propene (CCl₂═CClCH₂Cl or HCO-1230xa) into 2-chloro-3,3,3-trifluoro-1-propene (CF₃CCl═CH₂ or HCFO-1233xf) or 2,3,3,3-tetrafluoropropene (CF₃CF═CH₂ or HFO-1234yf), the conversion of 2,3,3,3-tetrachloro-1-propene (CCl₃CCl═CH₂ or HCO-1230xf) into 2-chloro-3,3,3-trifluoro-1-propene (CF₃CCl═CH₂ or HCFO-1233xf) or into 2,3,3,3-tetrafluoropropene (CF₃CF═CH₂ or HFO-1234yf), the conversion of 1-chloro-3,3,3-trifluoro-1-propene (CF₃CH═CHCl or HCFO-1233zd) or of 1,1,3,3-tetrachloro-1-propene (CCl₂═CHCHCl₂ or HCO-1230za) or of 1,3,3,3-tetrachloroprop-1-ene (CCl₃CH═CHCl or HCO-1230zd) into 1,3,3,3-tetrafluoropropene (CF₃CH═CHF or HFO-1234ze), the conversion of 2,3-dichloro-1,1,1-trifluoropropane (CF₃CHClCH₂Cl or HCFC-243db) into 2,3,3,3-tetrafluoropropene (CF₃CF═CH₂ or HFO-1234yf), the conversion of 2-chloro-1,1,1,2-tetrafluoropropane (CF₃CFClCH₃ or HCFC-244bb) into 1,1,1,2,2-pentafluoropropane (CF₃CF₂CH₃ or HFC-245cb), the conversion of 1,1,2,2-tetrachloroethylene (Cl₂C═CCl₂ or PER) into 1,1,1,2,2-pentafluoroethane (CF₃CF₂H or HFC-125), the conversion of 2-chloro-1,1,1-trifluoroethane (CF₃CH₂Cl or R-133a) into 1,1,1,2-tetrafluoroethane (CF₃CCH₂F or R-134a), the conversion of 1,1,2,2-tetrachloroethylene (Cl₂C═CCl₂ or PER) into 1,1,1,2-tetrafluoroethane (CF₃CCH₂F or R-134a), the conversion of 1,1,2-trichloroethylene (ClHC═CCl₂) into 1,1,1,2-tetrafluoroethane (CF₃CCH₂F or R-134a) and/or 1,1,1,2,2-pentafluoroethane (CF₃CF₂H or HFC-125).

More particularly, specific examples of fluorination reactions of compounds A include the conversion of 1,1,1,2,3-pentachloropropane (CCl₃CHClCH₂Cl or HCC-240db) into 2-chloro-3,3,3-trifluoro-1-propene (CF₃CCl═CH₂ or HCFO-1233xf), the conversion of 1,1,2,2,3-pentachloropropane (CHCl₂CCl₂CH₂Cl or HCC-240aa) into 2-chloro-3,3,3-trifluoro-1-propene (CF₃CCl═CH₂ or HCFO-1233xf), the conversion of 1,1,1,2,3-pentachloropropane (CCl₃CHClCH₂Cl or HCC-240db) into 2,3,3,3-tetrafluoropropene (CF₃CF═CH₂ or HFO-1234yf), the conversion of 1,1,2,2,3-pentachloropropane (CHCl₂CCl₂CH₂Cl or HCC-240aa) into 2,3,3,3-tetrafluoropropene (CF₃CF═CH₂ or HFO-1234yf), the conversion of 1,1,1,3,3-pentachloropropane (CCl₃CH₂CHCl₂ or HCC-240fa) into 1,3,3,3-tetrafluoropropene (CF₃CH═CHF or HFO-1234ze), the conversion of 1,1,2-trichloroethane (CHCl₂CH₂Cl or HCC-140) into 1-chloro-2,2-difluoroethane (CH₂ClCF₂H or HCFC-142), the conversion of 2-chloro-3,3,3-trifluoro-1-propene (CF₃CCl═CH₂ or HCFO-1233xf) into 2,3,3,3-tetrafluoropropene (CF₃CF═CH₂ or HFO-1234yf), the conversion of 1,1,2,3-tetrachloro-1-propene (CCl₂═CClCH₂Cl or HCO-1230xa) into 2-chloro-3,3,3-trifluoro-1-propene (CF₃CCl═CH₂ or HCFO-1233xf) or into 2,3,3,3-tetrafluoropropene (CF₃CF═CH₂ or HFO-1234yf), the conversion of 2,3,3,3-tetrachloro-1-propene (CCl₃Cl═CH₂ or HCO-1230xf) into 2-chloro-3,3,3-trifluoro-1-propene (CF₃CCl═CH₂ or HCFO-1233xf) or into 2,3,3,3-tetrafluoropropene (CF₃CF═CH₂ or HFO-1234yf), the conversion of 1-chloro-3,3,3-trifluoro-1-propene (CF₃CH═CHCl or HCFO-1233zd) or of 1,1,3,3-tetrachloro-1-propene (CCl₂═CHCHCl₂ or HCO-1230za) or of 1,3,3,3-tetrachloroprop-1-ene (CCl₃CH═CHCl or HCO-1230zd) into 1,3,3,3-tetrafluoropropene (CF₃CH═CHF or HFO-1234ze), the conversion of 1,2-dichloroethylene (CHCl═CClH or HCO-1130) into 1-chloro-2,2-difluoroethane (CH₂ClCF₂H or HCFC-142), the conversion of 2,3-dichloro-1,1,1-trifluoropropane (CF₃CHClCH₂Cl or HCFC-243db) into 2,3,3,3-tetrafluoropropene (CF₃CF═CH₂ or HFO-1234yf), the conversion of 2-chloro-1,1,1,2-tetrafluoropropane (CF₃CFClCH₃ or HCFC-244bb) into 1,1,1,2,2-pentafluoropropane (CF₃CF₂CH₃ or HFC-245cb), the conversion of 1,1,2,2-tetrachloroethylene (Cl₂C═CCl₂ or PER) into 1,1,1,2,2-pentafluoroethane (CF₃CF₂H or HFC-125), the conversion of 2-chloro-1,1,1-trifluoroethane (CF₃CH₂Cl or R-133a) into 1,1,1,2-tetrafluoroethane (CF₃CCH₂F or R-134a), the conversion of 1,1,2,2-tetrachloroethylene (Cl₂C═CCl₂ or PER) into 1,1,1,2-tetrafluoroethane (CF₃CCH₂F or R-134a), the conversion of 1,1,2-trichloroethylene (ClHC═CCl₂) into 1,1,1,2-tetrafluoroethane (CF₃CCH₂F or R-134a) and/or 1,1,1,2,2-pentafluoroethane (CF₃CF₂H or HFC-125).

According to another preferred embodiment, step a) may implement a gas-phase dehydrohalogenation reaction of a saturated C₁-C₄ hydrocarbon compound B comprising at least one halogen atom to form an unsaturated C₁-C₄ hydrocarbon compound and a hydrogen halide. Preferably, the compound B is selected from the group consisting of 1,1-difluoroethane, 1,1,1-trifluoroethane, 2-chloro-1,1,1-trifluoroethane, 1,1,1,2-tetrafluoroethane, 1,1,2,2-tetrafluoroethane, 1,1,1,2,2-pentafluoroethane, 1,1,1,2-tetrafluoropropane, 1,1,1,3,3-pentafluoropropane, 1,1,1,2,3,3-hexafluoropropane, 1,1,1,3,3,3-hexafluoropropane, 1,1,1,2,2,3-hexafluoropropane, 1,1,1,2,2-pentafluoropropane, 1,1,1,2,3-pentafluoropropane, 2,3-dichloro-1,1,1-trifluoropropane and 2-chloro-1,1,1,2-tetrafluoropropane. In particular, the compound B is selected from the group consisting of 1,1,1,2,2-pentafluoropropane, 1,1,1,3,3-pentafluoropropane, 2,3-dichloro-1,1,1-trifluoropropane and 2-chloro-1,1,1,2-tetrafluoropropane. Preferably, the hydrogen halide is HF or HCl.

Specific examples of gas-phase dehydrohalogenation of the compound B include the conversion of 1,1-difluoroethane (CHF₂CH₃ or HFC-152a) into vinyl chloride (CHF═CH₂ or HFO-1141), the conversion of 1,1,1-trifluoroethane (CF₃CH₃ or HFC-143a) into vinylidene fluoride (CF₂═CH₂ or HFO-1132a), the conversion of 2-chloro-1,1,1-trifluoroethane (CF₃CH₂Cl or HCFC-133a) into 2-chloro-1,1-difluoroethylene (CF₂═CHCl or HCFO-1122), the conversion of 1,1,1,2-tetrafluoroethane (CF₃CH₂F or HFC-134a) into trifluoroethylene (CF₂═CHF or HFO-1123), the conversion of 1,1,2,2-tetrafluoroethane (CHF₂CHF₂ or HFC-134) into trifluoroethylene (CF₂═CHF or HFO-1123), the conversion of 1,1,1,2-tetrafluoropropane (CH₃CHFCF₃ or HFC-254eb) into 1,1,1-trifluoropropene (CH₂═CHCF₃ or HFO-1243zf), the conversion of 1,1,1,3,3-pentafluoropropane (CHF₂CH₂CF₃ or HFC-245fa) into 1,3,3,3-tetrafluoropropene (CHF═CHCF₃ or HFO-1234ze), the conversion of 1,1,1,2,3,3-hexafluoropropane (CHF₂CHFCF₃ or HFC-236ea) into 1,2,3,3,3-pentafluoropropene (CHF═CFCF₃ or HFO-1225ye), the conversion of 1,1,1,3,3,3-hexafluoropropane (CF₃CH₂CF₃ or HFC-236fa) into 1,1,3,3,3-pentafluoropropene (CF₃CH═CF₂ or HFO-1225zc), the conversion of 1,1,1,2,2,3-hexafluoropropane (CF₃CF₂CFH₂ or HFC-236cb) into 1,2,3,3,3-pentafluoropropene (CHF═CFCF₃ or HFO-1225ye), the conversion of 1,1,1,2,2-pentafluoropropane (CF₃CF₂CH₃ or HFC-245cb) into 2,3,3,3-tetrafluoropropene (CF₃CF═CH₂ or HFO-1234yf) and the conversion of 1,1,1,2,3-pentafluoropropane (CF₃CHFCH₂F or HFC-245eb) into 2,3,3,3-tetrafluoropropene (CF₃CF═CH₂ or HFO-1234yf), the conversion of (CF₃CHClCH₂Cl or HCFC-243db) into 2-chloro-3,3,3-trifluoro-1-propene (CF₃CCl═CH₂ or HCFO-1233xf), the conversion of 2-chloro-1,1,1,2-tetrafluoropropane (CF₃CFClCH₃ or HCFC-244bb) into 2,3,3,3-tetrafluoropropene (CF₃CF═CH₂ or HFO-1234yf).

The catalytic bed temperature T1 may be between 100° C. and 500° C., advantageously between 150° C. and 450° C., preferably between 200° C. and 400° C., more preferentially between 250° C. and 380° C.

Step a) may also be implemented according to the following operating conditions:
an HF/hydrocarbon compound molar ratio between 1:1 and 150:1, preferably between 2:1 and 125:1, more preferentially between 3:1 and 100:1;
a contact time between 1 and 100 s, preferably between 2 and 75 s, in particular between 3 and 50 s;
a pressure between atmospheric pressure and 20 bar, preferably between 2 and 18 bar, more preferentially between 3 and 15 bar.

Those skilled in the art will adapt the operating conditions above according to the reaction to be carried out in step a).

Step a) may be implemented over a duration of between 2000 and 25 000 h, preferably between 2500 and 24 000 h, more preferentially between 3000 and 20 000 h.

An oxidant, such as oxygen or chlorine, may be added during step a). The molar ratio of the oxidant to the compound A or B may be between 0.005 and 2, preferably between 0.01 and 1.5. The oxidant may be pure oxygen, air or a mixture of oxygen and nitrogen.

Step a) may optionally comprise a step of regeneration of the catalyst in alternation with said catalytic reaction. The regeneration step is generally implemented in the presence of a stream comprising oxygen at a temperature of between 100° C. and 500° C.

At the end of the implementation of step a), the catalyst is subjected to step b) according to the process of the present invention.

Step b)

According to the present process, step b) comprises causing an inert gas to flow through the catalytic bed. Preferably, step b) is implemented at a catalytic bed temperature T2 that is lower than T1. Thus, it is not necessary to heat the catalytic bed to remove the hydrogen halide present in the reactor or the catalyst. In order to maximize the removal of the hydrogen halide, the catalytic bed temperature T2 is greater than 30° C. Thus, at the start of implementation of step b), the temperature T2 is greater than 30° C.

After the implementation of step a), the stream of reactants is stopped, the temperature of the catalytic bed decreases from the temperature T1 to the temperature T2, which is lower than T1, and the inert gas is introduced into the reactor.

Preferably, the catalytic bed temperature T2 is greater than 40° C., advantageously greater than 50° C., preferably greater than 60° C., more preferentially greater than 70° C., in particular greater than 80° C., more particularly greater than 90° C., favorably greater than 100° C.

Preferably, the catalytic bed temperature T2 is lower than 380° C., advantageously lower than 360° C., preferably lower than 340° C., more preferentially lower than 320° C., in particular lower than 300° C., favorably lower than 250° C.

According to a preferred embodiment, the inert gas is introduced into the reactor at a temperature of between ambient temperature and T2, advantageously of between ambient temperature and 50° C., and in particular the inert gas is introduced into the reactor at ambient temperature.

The passage of the inert gas through the catalytic bed leads to a decrease in the catalytic bed temperature T2 during the implementation of step b). Preferably, the temperature T2 decreases at a rate of less than 5° C./min during the implementation of step b), in particular of less than 1° C./min, during the implementation of step b).

According to a preferred embodiment, the inert gas flows at a flow rate of greater than 0.1 ml/min per ml of catalyst, advantageously of greater than 0.2 ml/min per ml of catalyst, preferably of greater than 0.3 ml/min per ml of catalyst, more preferentially of greater than 0.4 ml/min per ml of catalyst, in particular of greater than 0.5 ml/min per ml of catalyst, favorably of greater than 0.6 ml/min per ml of catalyst, advantageously favorably of greater than 0.7 ml/min per ml of catalyst, preferentially favorably of greater than 0.8 ml/min per ml of catalyst, particularly favorably of greater than 0.9 ml/min per ml of catalyst.

According to a preferred embodiment, step b) comprises a step b1) of cooling the catalytic bed from the temperature T1 to T2, and then a step b2) of causing said inert gas to flow through the catalytic bed.

Preferably, the inert gas is nitrogen or argon. In particular nitrogen.

Preferably, at the outlet of the reactor, the inert gas contains a content by mass of CO and CO2 of less than 100 ppm.

### Examples

The apparatus used comprises a tubular reactor made from INCONEL© 600 (internal diameter of 28 mm−length=600 mm), placed vertically in an electric tube furnace. The reactor is equipped with indicators for pressure and temperature (movable thermocouple in an Inconel sleeve placed coaxially at the center of the tube). The fixed catalytic bed consists of a lower layer of corundum followed by a 180 ml layer of catalyst and an upper layer of corundum. The catalyst used is an Ni—Cr/AlF₃ catalyst. Before use, it is dried and then activated in the presence of a mixture of hydrofluoric acid and nitrogen, at a temperature of between T=320° C. and T=350° C.

The characteristics of the catalyst after activation are as follows:
BET surface area: 38.78 m²/g;
chemical composition: Al: 19.0%, F: 61.2%, Cr: 4.5%, Ni: 4.4%

The reactants are introduced continuously at the upper end of the reactor and preheated to the furnace temperature through the upper layer of corundum, the gaseous products of the reaction exit at the lower end of the reactor through a pressure-regulating valve; the gas stream exiting from the valve is analyzed by gas chromatography.

Test 1 (Invention)

The reaction is conducted at atmospheric pressure and at a temperature of T=350° C. by continuously supplying anhydrous HF (137.6 g·h⁻¹) and perchloroethylene (28.3 g·h⁻¹). The GHSV (gas hourly space velocity) is 2000 h⁻¹. The HF:organic molar ratio is 40.3.

After 19 h of reaction, the composition of the organic stream exiting the reactor is given in table 1 below.

TABLE 1

| Composition | PER | F125 | F124 | F123 | F121 + F122 | Others |
|---|---|---|---|---|---|---|
| mol % | 29.39 | 21.68 | 25.33 | 13.55 | 4.77 | 5.28 |

After 98 h of reaction, the introduction of the reactants and the heating of the electric furnace are stopped. Nitrogen is introduced into the reactor at a flow rate of 10 l·h⁻¹ (0.9 ml·min⁻¹ per ml of catalyst).

After flushing for 17 h, the temperature in the reactor is T=140° C. (i.e. a decrease at an average rate of 12° C. per hour).

Test 2 (Comparative)

A test identical to test 1 (same batch of activated catalyst) is carried out until the stopping of the reactants. After 98 h of reaction, the introduction of the reactants is stopped and the heating of the electric furnace is increased until a temperature of 360° C. is reached. Nitrogen is introduced into the reactor at a flow rate of 10 l·h⁻¹ (0.9 ml·min⁻¹ per ml of catalyst). The temperature of the furnace is maintained at a temperature of T=360° C. for 8 hours.

### Example 1

The catalyst of test 1 and the catalyst of test 2 are regenerated in identical fashion at atmospheric pressure, by treatment in air (1.5 l·h⁻¹) at a temperature of T=350° C. for 72 hours.

After regeneration, the catalysts are analyzed (table 2).

TABLE 2
BET surface area (m²/g)

| Catalyst of test 1 | Catalyst of test 2 |
|---|---|
| (after regeneration) | (after regeneration) |
| 25.79 | 20.03 |

The catalysts of test 1 and of test 2 thus regenerated are tested in the fluorination reaction of perchloroethylene. The reaction is conducted at atmospheric pressure and at a temperature of T=350° C. by continuously supplying anhydrous HF (137.6 g·h⁻¹) and perchloroethylene (28.3 g·h⁻¹). The GHSV (gas hourly space velocity) is 2000 h⁻¹. The HF:organic molar ratio is 40.3. The analysis of the composition of the organic stream exiting the reactor is also conducted after 19 h and 43 h of reaction. The comparative results are presented in table 3 below.

TABLE 3

| | | | | | | F121 + | |
|---|---|---|---|---|---|---|---|
| Composition (mol %) | | PER | F125 | F124 | F123 | F122 | Others |
| Catalyst of | 19 h | 37.97 | 10.08 | 23.56 | 16.05 | 5.59 | 6.75 |
| test 1 | 43 h | 40.45 | 13.03 | 21.99 | 12.96 | 5.94 | 5.63 |
| Catalyst of | 19 h | 45.89 | 8.04 | 17.19 | 14.95 | 6.48 | 7.45 |
| test 2 | 43 h | 48.2 | 8.62 | 15.19 | 13.99 | 6.5 | 7.5 |

These results clearly show that the treatment conducted on the catalyst of test 2 is harmful to it (lower BET surface area and catalytic activity). In contrast, the nitrogen treatment conducted on the catalyst of the test 1 makes it possible to obtain a better catalytic activity thereof after regeneration. Step b) according to the present invention makes it possible to avoid the premature degradation of the catalyst and to thus obtain a catalyst that is more effective later on when it is reused, for example after regeneration.

1-14. (canceled)

15. A process for treating, in a reactor containing a catalytic bed, a solid catalyst, said process comprising the steps of:
a) implementing, in said reactor, a gas-phase catalytic reaction at a catalytic bed temperature T1 in the presence of a hydrogen halide or giving rise to the formation of a hydrogen halide, and
b) causing an inert gas to flow through the catalytic bed at a catalytic bed temperature T2 that is lower than T1, the temperature T2 being greater than 30° C.

16. The process as claimed in claim 15, characterized in that the inert gas introduced into the reactor is at a temperature of between ambient temperature and the temperature T2.

17. The process as claimed in claim 15, characterized in that the inert gas introduced into the reactor is at ambient temperature.

18. The process as claimed in claim 15, characterized in that the hydrogen halide is hydrogen fluoride or hydrogen chloride.

19. The process as claimed in claim 15, characterized in that the hydrogen halide is in anhydrous form.

20. The process as claimed in claim 15, characterized in that the temperature T2 decreases during the implementation of step b).

21. The process as claimed in claim 15, characterized in that the temperature T2 decreases at a rate of less than 1° C./min during the implementation of step b).

22. The process as claimed in claim 15, characterized in that the inert gas flows at a flow rate of greater than 0.1 ml/min per ml of catalyst.

23. The process as claimed in claim 15, characterized in that the catalyst is based on carbon or based on a metal selected from the group consisting of Cr, Fe, Sb, Ni, Co, Zn, Al and Mn.

24. The process as claimed in claim 15, characterized in that step a) implements a gas-phase reaction between HF and a C₁-C₄ halohydrocarbon compound A or step a) implements a gas-phase dehydrohalogenation reaction of a saturated C₁-C₄ hydrocarbon compound B comprising at least one halogen atom to form an unsaturated C₁-C₄ hydrocarbon compound and a hydrogen halide.

25. The process as claimed in claim 15, characterized in that the compound A is selected from the group consisting of 1,1,2-trichloroethane, 2-chloro-1,1,1-trifluoroethane, 1-chloro-1,1,2-trifluoroethane, 1-chloro-1,2,2-trifluoroethane, 1,1,1,3,3,3-hexachlorodifluoropropane, 1,1,1,3,3,3-hexachloropropane, 1,1,1,3,3-pentachloropropane, 2,2,3-trichloro-1,1,1,3,3-pentafluoropropane, 1,1,1,3,3,3-hexachlorodifluoropropane, 1,1-dichloro-2,2,3,3,3-pentafluoropropane, 1,3-dichloro-1,2,2,3,3-pentafluoropropane, 1,1,1,2,3-pentachloropropane, 1,1,2,2,3-pentachloropropane, 1,1,1,3,3-pentachloropropane, 1,2-dichloroethylene, 1,1,2-trichloroethylene, 1,1,2,2-tetrachloroethylene, 1,1,2-trichloro-3,3,3-trifluoropropene, hexafluoropropene, 1,1,3,3,3-pentafluoropropene, 1,3,3,3-tetrafluoropropene, 2-chloro-3,3,3-trifluoropropene, 1,1,2,3-tetrachloropropene, 2,3,3,3-tetrachloropropene, 1-chloro-3,3,3-trifluoropropene, 1,1,3,3-tetrachloropropene, 1,3,3,3-tetrachloropropene, 2,3-dichloro-1,1,1-trifluoropropane, and 2-chloro-1,1,1,2-tetrafluoropropane; or
the compound B is selected from the group consisting of 1,1-difluoroethane, 1,1,1-trifluoroethane, 2-chloro-1,1,1-trifluoroethane, 1,1,1,2-tetrafluoroethane, 1,1,2,2-tetrafluoroethane, 1,1,1,2,2-pentafluoroethane, 1,1,1,2-tetrafluoropropane, 1,1,1,2,3,3-hexafluoropropane, 1,1,1,3,3,3-hexafluoropropane, 1,1,1,2,2,3-hexafluoropropane, 1,1,1,2,2-pentafluoropropane, 1,1,1,2,3-pentafluoropropane, 1,1,1,3,3-pentafluoropropane, 2,3-dichloro-1,1,1-trifluoropropane, and 2-chloro-1,1,1,2-tetrafluoropropane.

26. The process as claimed in claim 15, characterized in that the process comprises a step c) of unloading the catalyst from said reactor.

27. The process as claimed in claim 15, characterized in that step b) comprises a step b1) of cooling the catalytic bed from the temperature T1 to T2, and then a step b2) of causing said inert gas to flow through the catalytic bed.

28. A process for treating, in a reactor containing a catalytic bed, a solid catalyst, said process comprising the steps of:
a) implementing, in said reactor, a gas-phase catalytic reaction at a catalytic bed temperature T1 in the presence of a hydrogen halide or giving rise to the formation of a hydrogen halide, and
b) causing an inert gas to flow through the catalytic bed at a catalytic bed temperature T2 that is lower than T1, the temperature T2 decreasing during the implementation of step b).
